(19) United States
(12) Patent Application Publication
(10) Pub. No.: US 2022/0058581 A1
(43) Pub. Date: Feb. 24, 2022

(54) COMPUTER-IMPLEMENTED SYSTEM AND METHOD FOR ASSOCIATING PRESCRIPTION DATA AND DE-DUPLICATION

(71) Applicant: SOURCE HEALTHCARE ANALYTICS, LLC, Phoenix, AZ (US)

(72) Inventors: PETER C. DEMOGENES, Scottsdale, AZ (US); Karin Chun Hayes, Mesa, AZ (US); Jeffrey Little, Tempe, AZ (US); Keith Sheridan, East Greenwich, RI (US); Paulette Weidman, Peoria, AZ (US)

(21) Appl. No.: 17/519,041

(22) Filed: Nov. 4, 2021

Related U.S. Application Data
(63) Continuation of application No. 14/973,065, filed on Dec. 17, 2015, now abandoned.
Application No. 14/973,065 is a continuation of application No. 13/008,102, filed on Jan. 18, 2011, now abandoned.

(51) Int. Cl. G06Q 10/10; G16H 20/10

(52) U.S. Cl. CPC G06Q 10/10; G16H 20/10

(57) ABSTRACT

A prescription association system and method for grouping together prescription-related transactions and creating groups or “clusters” of prescriptions having similar characteristics. Through an association process, the prescription cluster describes the events surrounding prescription activity. This includes prescribing patterns, payer influences, and patient acceptance of therapy. The same prescription transaction from one data provider may contain additional or different information that can enhance a corresponding duplicate transaction or set of claim lifecycle transactions from another provider. The disclosed processes create unique linking across claims, payers, and patients and form the basis for relating and measuring payer, patient, practitioner, and pharmaceutical promotion influences on healthcare utilization and treatment.

### CROSS-REFERENCE TO RELATED APPLICATIONS

This application is a continuation of U.S. patent application Ser. No. 14/973,065, filed Dec. 17, 2015, which is a continuation of U.S. patent application Ser. No. 13/008,102, filed Jan. 18, 2011, the contents of which are incorporated herein by reference in their entirety.

### TECHNICAL FIELD

The present invention relates to computer-based processes for associating prescription-related information. More specifically, the present disclosure is directed to a system and method of analytics relating to pharmaceutical prescriptions including the interaction among physicians, patients and payers.

### BACKGROUND INFORMATION

Due to the dynamic nature of the healthcare industry, it is difficult to reliably track and predict provider, payer and patient behavior with respect to prescription transactions. Factors such as government and commercial payers' influence on brand performance and patients' decisions to switch to generic prescriptions create challenges to determining the percentage of patients who will continue to refill given prescriptions on a timely basis. As a result, a system of data processing systems and techniques for determining factors that influence prescription use and transactions would greatly assist organizations in the industry to make optimal business decisions. Such a system would need to take many factors into account to accurately analyze marketplace influencers.

For example, there are a number of challenges presented by patient behavior patterns that can complicate the association of prescription-related data. A patient may call in or drop off a prescription, but that patient may delay picking up that prescription or not pick up the prescription at all (called a reversal). In other cases, a patient may end up paying for a partial prescription fill, or receive two refills at once, for instance, if the patient will be leaving on vacation.

With respect to insurance providers, a provider normally controls a patient's access to prescription medications through the adjudication process. For example, if a prescribed drug is not on the insurer's formulary, any claim for that prescription from a pharmacy will be returned as a rejection. At this point, the patient may ultimately choose to not purchase the medication under these circumstances. The prescription might also be: (a) resubmitted using a different drug that is acceptable to the healthcare plan provider, such as a generic version; (b) covered, but at a much higher cost for the patient; and/or (c) submitted as a special request to get the drug approved at a lower price due to medical necessity.

With respect to pharmacy influences, each pharmacy or chain may have unique policies and procedures for handling events such as returning product to the shelf, reusing prescription numbers, assigning patient IDs, and providing prescription information to external services. Further, these policies are often subject to change, and such a policy change may result in a prescription that was formerly honored now being reversed or voided. If a pharmacy merges with another chain, often the business rules of the acquiring chain are then applied to the acquired pharmacies, and patient information is transitioned to the acquiring chain's systems. This can cause data inconsistencies that are not easily resolved.

Physicians are motivated to write prescriptions that are filled at the pharmacy with minimum callbacks. A callback occurs when a healthcare plan or patient refuses to pay for a prescription. Since physicians do not typically know co-pay information or which drugs are approved under a patient's healthcare plan, they typically make educated guesses when writing prescriptions. When a formulary change in one healthcare plan leads to callbacks, a physician may decide to stop prescribing a certain drug for that patient. This formulary change in one healthcare plan might also influence this drug product's sales performance across the rest of the particular physician's patients' healthcare plans, creating a “spillover” in this physician's prescribing behavior. This spillover should also be accounted for in any marketplace influencer analysis.

Current systems have limited abilities to analyze data and often suffer from at least some the following deficiencies:

Dealing with inconsistent layouts: a “source file” contains relevant data from a particular source of prescription information. Source files can be provided by, for example, a pharmacy or service bureau. Not all source files have the same layouts, and consequently inconsistencies between source files can lead to errors during data processing. For example, some sources provide transaction information in accordance with an industry standard layout from the National Council for Prescription Drug Programs (“NCPDP”). However, source files provided by outside suppliers such as pharmacy chains and service bureaus typically have custom layouts defined in contractual agreements. In order to provide an accurate market analysis, related transactions reported by these different sources would have to be associated. For example, information regarding a prescription written by a physician would have to be associated with information related to the patient ordering and filling that prescription, even if this information was provided by different sources. However, the inconsistency between source file layouts makes it difficult to associate data from related transactions and often makes it necessary to transform all received data files to a set of standard values in order to do so. Further complicating the matter is the fact that a single transaction is often reflected in multiple source files, creating duplicate data that would have to be recognized to prevent inaccurate data entries.

Attribute availability and formatting: An “attribute” is an element of information related to a transaction that is provided in a source file. Examples of attributes include a prescribing physician's name and/or the prescription number. Not all sources will provide the same attributes, and sometimes sources will use different labels to refer to the same attribute. For example, one source may identify a transaction by a prescriber DEA number, which is a unique identifier for anyone who can prescribe medication, while another source may identify the transaction by an NPI number, which is an identifier for a health care provider under the National Plan & Provider Enumeration System (“NPPES”). As another example, one source may provide a refill code for a transaction, while another will provide a refill number. A source may also remove or revise a provided attribute without notice.

Data encryption: Attributes provided by a source may be encrypted, either with or without repeatable passwords, making some of the information unavailable to third parties. In the event that a particular attribute is unavailable, additional methodologies are needed to process the transactions using only the limited available data.

Disparate receipt dates: Data related to the same prescription but provided by different sources may be received at different times. This necessitates the use of a flexible or rolling time period to handle associated transactions that are received over a period of time. For example, some sources typically send prescription claim data the day after the activity takes place. However, data provided by pharmacies and chain stores may lag by a week or more.

Limited ability to cluster prescription events: An “event cluster” is a set of one or more transactions related to a specific prescription event. For example, a physician writes a prescription for a patient who then fills the prescription at a pharmacy. Each of these actions, or events, might be reported as a transaction by different data providers, but because they relate to the same prescription event they should be associated in one event cluster. Moreover, since not every data provider will provide every desired attribute for each transaction, when the information from multiple data providers is associated in an event cluster, a more complete picture of the prescription event is formed. For example, if a first transaction is missing an attribute (e.g., date of birth or allergies), but a second associated transaction is able to provide the missing attribute (e.g., the time the prescription was refilled), the event cluster would combine the data from each transaction to provide a more detailed picture of the prescription event. An event cluster may include an array of attributes, including, for example, patient demographics (e.g., date of birth, race, gender, allergies, etc.), patient identification numbers, prescription history, medical records, life-cycle information, and/or other data relevant to drug prescriptions. Current systems are incapable of, or are limited in, associating transactions in different source files to the same event cluster.

Accordingly, there is a need to have a methodology in place designed to identify these conditions and take into consideration the differences, inconsistencies and limitations of prescription data to properly analyze the extent to which the patient, healthcare plan and provider affect the manner in which a medication is filled and eventually paid or not paid for by the patient. Normally, this process is complex due to the need to interpret patterns of behavior based on sequences of transactions, and the need to identify the duplicate associated transactions. Without this process in place, the result will be an overstatement or understatement of prescription activity, inaccurate analysis and reporting results.

### SUMMARY

Various embodiments are disclosed herein for overcoming one or more of the aforementioned deficiencies. Accordingly, a process for analyzing market influences comprising a multi-pass algorithm for identifying duplicate transactions and creating groups or “event clusters” of transactions related to the same prescription event is described. Through the association process, the event cluster contains the transactions surrounding the prescription event. This includes prescribing patterns, payer influences and patient acceptance of therapy. The same transaction from one data provider may contain additional or different information that can enhance a corresponding transaction from another provider. For example, transactions in the source files from the payer adjudication process may have “life-cycle information” about the influence of the payer on the brand but may be missing the prescriber identifiers. A corresponding source file from another source may contain the prescriber identifiers, thus creating a more complete view of the prescription event.

According to a first aspect of the present invention, a processor-implemented method for associating prescription-related data comprises the step of providing at least one processor configured to: receive batch transaction data comprising a plurality of predetermined classifications; process the classifications in order to obtain attributes for the classifications; process the attributes to determine matches among the attributes for a respective classification for a predetermined period of time; and group into one or more clusters the matches for each attribute during the predetermined period of time to identify events associated with the prescription-related data.

According to a second aspect of the present invention, a computer system for associating prescription-related data comprises a memory; a communication device operatively coupled to the memory to receive batch transaction data relating to the prescription-related data, said batch transaction data comprising a plurality of predetermined classifications; and at least one processor, operatively coupled to the communications device for processing the classifications in order to obtain attributes for the classifications and processing the attributes to determine matches among the attributes for a respective classification for a predetermined period of time wherein the at least one processor groups the matches for each attribute during the predetermined period of time to identify events associated with the prescription-related data.

According to a third aspect of the present invention, a computer network for associating prescription-related data comprises at least one memory device; at least one communication device operatively coupled to the at least one memory device to receive batch transaction data relating to the prescription-related data, said batch transaction data comprising a plurality of predetermined classifications; and at least one processor, operatively coupled to the communications device for processing the classifications in order to obtain attributes for the classifications and processing the attributes to determine matches among the attributes for a respective classification for a predetermined period of time; wherein the at least one processor (i) groups the matches for each attribute during the predetermined period of time to identify events associated with the prescription-related data, (ii) processes the attributes by identifying a first matching attribute comprising pharmacy identification, prescription number, refill code, and a prescription fill date within a specified range, and (iii) processes the attributes by identifying a second matching attributes comprising pharmacy identification, prescription number, drug identification code, and a prescription fill date within a specified range.

For this application the following terms and definitions shall apply:

The terms “communicate” and “communicating” as used herein include both conveying data from a source to a destination and delivering data to a communications medium, system, channel, network, device, wire, cable, fiber, circuit and/or link to be conveyed to a destination, and the term “communication” as used herein means data so conveyed or delivered. The term “communications” as used herein includes one or more of a communications medium, system, channel, network, device, wire, cable, fiber, circuit and link.

The terms “coupled,” “coupled to,” and “coupled with” as used herein each mean a relationship between or among two or more devices, apparatus, files, circuits, elements, functions, operations, processes, programs, media, components, networks, systems, subsystems, and/or means, constituting any one or more of (a) a connection, whether direct or through one or more other devices, apparatus, files, circuits, elements, functions, operations, processes, programs, media, components, networks, systems, subsystems, or means, (b) a communications relationship, whether direct or through one or more other devices, apparatus, files, circuits, elements, functions, operations, processes, programs, media, components, networks, systems, subsystems, or means, and/or (c) a functional relationship in which the operation of any one or more devices, apparatus, files, circuits, elements, functions, operations, processes, programs, media, components, networks, systems, subsystems, or means depends, in whole or in part, on the operation of any one or more others thereof.

The term “data” as used herein means any indicia, signals, marks, symbols, domains, symbol sets, representations, and any other physical form or forms representing information, whether permanent or temporary, whether visible, audible, acoustic, electric, magnetic, electromagnetic or otherwise manifested. The term “data” as used to represent predetermined information in one physical form shall be deemed to encompass any and all representations of corresponding information in a different physical form or forms.

The term “database” as used herein means an organized body of related data, regardless of the manner in which the data or the organized body thereof is represented. For example, the organized body of related data may be in the form of one or more of a table, a map, a grid, a packet, a datagram, a frame, a file, an e-mail, a message, a document, a report, a list or in any other form.

The term “network” as used herein includes both networks and inter-networks of all kinds, including the Internet, and is not limited to any particular network or inter-network.

The term “processor” as used herein means processing devices, apparatus, programs, circuits, components, systems and subsystems, whether implemented in hardware, tangibly embodied software or both, and whether or not programmable. The term “processor” as used herein includes, but is not limited to, one or more computers, hardwired circuits, signal modifying devices and systems, devices and machines for controlling systems, central processing units, programmable devices and systems, field-programmable gate arrays, application-specific integrated circuits, systems on a chip, systems comprising discrete elements and/or circuits, state machines, virtual machines, data processors, processing facilities and combinations of any of the foregoing.

The present disclosure illustrates systems and methods by which processes create unique linking across claims, payers and patients and form the basis for relating and measuring payer, patient, practitioner and pharmaceutical influences on healthcare utilization and treatment.

### BRIEF DESCRIPTION OF THE DRAWINGS

Embodiments of the present invention are illustrated by way of example and not limitation in the figures of the accompanying drawings, in which like references indicate similar elements and in which:

FIG. 1A illustrates a portion of an exemplary method for associating prescription-related data under an embodiment of the invention;

FIG. 1B illustrates a further step of the exemplary method for associating prescription-related data;

FIG. 2 shows the structure of tables that can be used to store information according to an embodiment of the invention;

FIG. 3 is an example of a system that executes the processes of FIGS. 1A-1B in accordance with an embodiment of the present invention.

### DETAILED DESCRIPTION

The present invention is described herein with reference to one or more exemplary embodiments, however, it should be understood that the present invention is not limited to these embodiments. Those skilled in the art will appreciate that other arrangements, formulations and other elements can be used instead, and some elements may be omitted altogether. In the following description, well-known functions or constructions may not be described in detail because they would obscure the invention in unnecessary detail.

Under an exemplary embodiment, a computer-implemented process is designed to receive daily prescription transactional data and associate it with prescription transactional data received at a different time and/or from a separate source. Daily prescription transactional data can be provided, for example, by source files. The process can also be adapted to properly sequence the transactions within an event cluster, and to identify a final state of the event cluster.

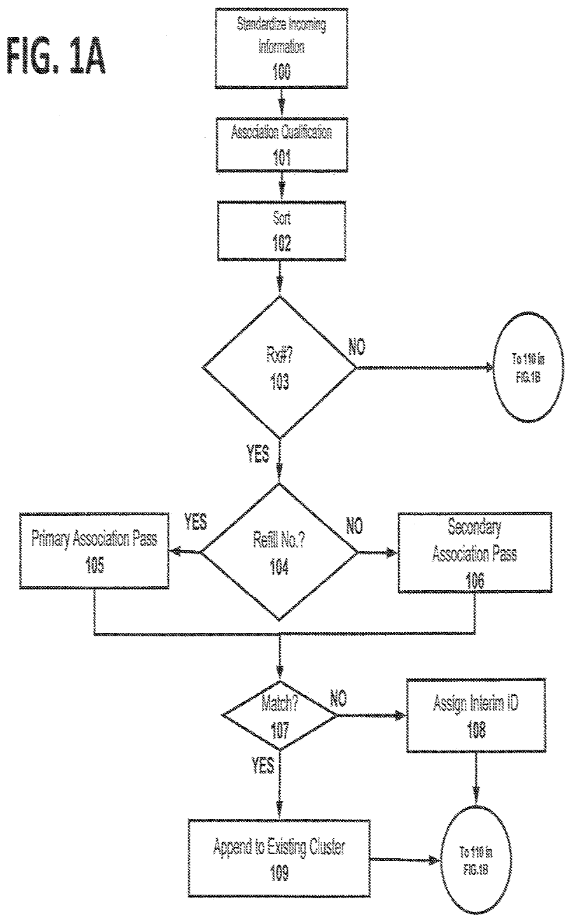

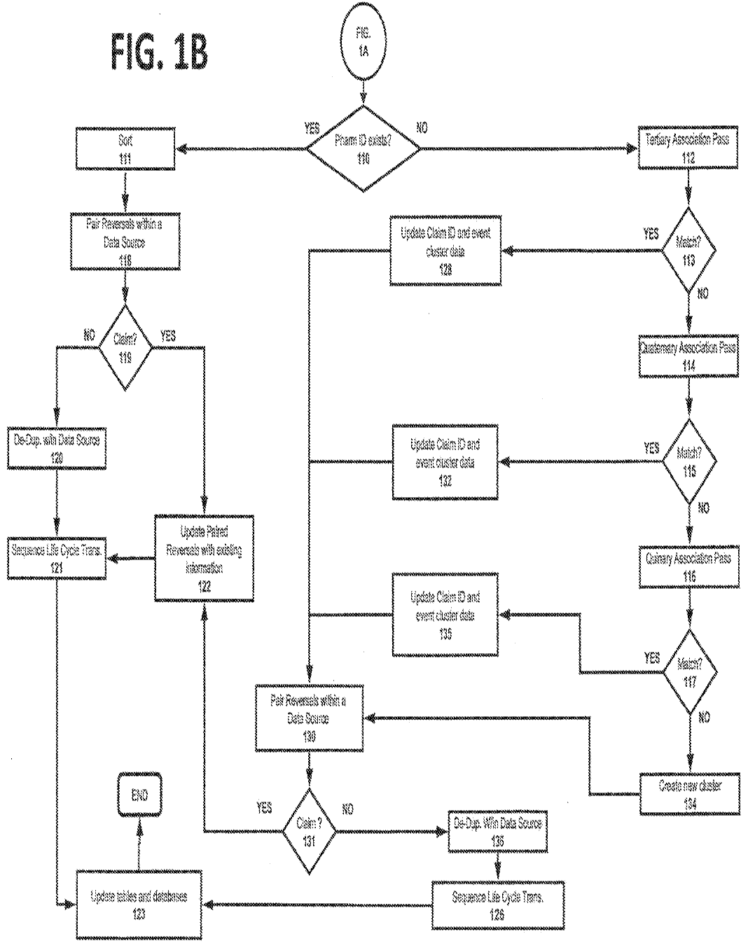

In FIG. 1A, an exemplary process for identifying and processing transactions for inclusion in an event cluster according to an embodiment of the invention is described. Identification of transactions requires the use of attributes that are preferably obtained from an Operational Data Store. Operational Data Store (or “ODS”) is preferably a database for integrating data from multiple sources to make analysis of the information more efficient. Because the data originates from multiple sources, the ODS can be adapted to standardize the data so that data from multiple sources can be analyzed together 100. This standardization may involve truncating extraneous digits or resolving redundancies. The data may be standardized according to one or more predetermined methods. For example, characters may be capitalized/dropped to lowercase, hidden characters/symbols may be removed and/or, depending on the attribute, the one or more characters may be justified (e.g., aligned to the left, right, center, top, bottom, and/or middle). In certain numeric/alphanumeric situations, decimal places may be adjusted to a standard format (e.g., to 2 decimals spaces for US currency), spacing may be adjusted and/or null values may be maintained as “null” to avoid confusion with a zero value. Similarly, when multiple sources use different code formats, a cross-reference method may be implemented to standardize all codes into a single format. Other procedures to standardize data types may be required depending on the format of the data delivered by a particular source, and are within the scope and spirit of the invention.

According to one embodiment of the present invention, transactions to be processed for association in an event cluster are selected from the set of standardized transaction at step 101. Transactions may be selected based on a number of criteria or parameters, including, for example, transactions from a specific source, transactions containing specific attributes, transactions within a specific time frame, and/or transactions related to a particular pharmaceutical. Other combinations of selecting criteria to determine qualified transactions are within the scope and spirit of the invention.

According to another embodiment of the present invention, the data from the selected transactions is stored in a temporary table for a specified amount of time, such as 60 days, so that subsequent related transactions occurring within the specified time frame can modify this information before it is loaded into a more permanent database. In an embodiment of the invention, a separate data warehouse is used to store other data that is generally used on a less frequent basis.

In a preferred embodiment, the transactions selected to be processed by the exemplary steps in FIG. 1A are all provided by the same source and all contain pharmacy IDs. There are a number of possible sources, including, for example, pharmacies (e.g., a pharmacy franchise/chain), clearing houses, and/or service bureaus (e.g., an entity that collects point of service pharmaceutical data from multiple sources). Data from multiple sources is combined and processed in the exemplary steps in FIG. 1B.

The selected transaction data may be sorted based on selected attributes 102 in order to accelerate data processing. Selected attributes can be, for example, the source of data, the pharmacy where the prescription was filled, a prescription number, the date the prescription was filled, the date the transaction was received and processed, and/or the status of the transaction. When a set of data is sorted by a specific attribute, it can be referred to as being “anchored” by that attribute. In a preferred embodiment of the present invention, all transactional data provided by a single source is anchored by pharmacy ID at step 102. It is understood by those skilled in the art that other attributes may be selected or used for the sorting process as well. Sorting step 102 is not a required step, but it may be implemented to accelerate the processing of the information.

According to an embodiment of the invention, in order to match the incoming transaction with an existing event cluster, selected attributes in a transaction selected for association at step 101 are compared with attributes in event clusters already in the system. As discussed in greater detail below, if the selected attributes from the transaction match those in the event cluster, the incoming transaction is associated with that transaction cluster. If there is no match, the incoming transaction becomes a new event cluster. Each time a selection of attributes is compared to the attributes in existing event clusters, it is referred to as an “association pass”.

According to an embodiment of the invention, association passes that occur earlier in the process have greater potential for accurate event cluster association than later association passes. For example, if attributes compared during the primary association pass match, there is a higher certainty that the transactions should be part of the same event cluster than if the attributes compared during the quinary association pass match. While the embodiments described in this specification specify the attributes to be compared during each association pass, those skilled in the art will recognize that comparing different attributes at association passes, as well as changing the number of association passes, is within the scope and spirit of the invention.

At step 103, if the transaction contains a pharmacy ID and a prescription number, then the process proceeds on to step 104. If the transaction does not contain a pharmacy ID and a prescription number, then the process skips to step 110 in FIG. 1B as discussed below.

At step 104, if the transaction contains a refill number, then a primary association pass on the transaction is run on the incoming transaction at step 105. If this attribute is not present, then a secondary association pass at step 106 is run.

At primary association pass 105, the pharmacy ID, prescription number, prescription fill date, and refill code of the incoming transaction are compared to the same attributes in the currently-existing event clusters to determine if the incoming transaction matches any of those event clusters. A prescription number is a unique number within a particular pharmacy's system that the pharmacy assigns to a prescription when it is filled. It may also be referred to as “Rx #”. A refill code is a number that may be associated with a transaction to indicate if the prescription is a refill, and if so, which refill the prescription represents. For example, a refill code value of 3 may identify the transaction as the third refill for a particular subscription. As another example, a null value or other designated value such as “99” may indicate that the prescription is a new prescription. In primary association pass 105, if the prescription number and refill code match, the incoming transaction will be associated with the matched event cluster, since the match indicates it is the same transaction.

In an alternate embodiment, the transactions do not have to be anchored by any attribute and the association pass comparisons can be made without an anchor.

In secondary association pass 106, the pharmacy ID, prescription number, first 10 digits of the National Drug Code (“NDC”) and prescription fill date of the incoming transaction are compared to the same attributes in existing event clusters to determine whether the transaction should be associated with that event cluster. The NDC number is typically a unique 10-digit, 3-segment numeric identifier assigned to each medication used to identify the labeler or vendor, labeler product, and trade package. If these attributes match, the transaction will be associated as part of the same event cluster.

If a match has occurred 107 in either the primary 105 or secondary 106 association passes, then the update and append process 109 updates the event cluster to include the information from the incoming transaction. If no match is found in either primary association pass 105 or secondary association pass 106, then a temporary new event cluster ID for the incoming prescription transaction, called an interim ID, is created 108. After step 108 or step 109, the next step is step 110 in FIG. 1B. In an alternative embodiment, no interim ID is created at 109. Therefore, if step 107 results in a “no”, then the process skips directly to step 110.

FIG. 1B illustrates a further step of the exemplary method for determining whether data from an incoming prescription transaction should be associated with an existing event cluster according to an embodiment of the invention. If desired, FIG. 1B can be run at different temporal intervals than FIG. 1A. For example, the process illustrated in FIG. 1A may be run regularly on incoming transactions throughout the day, but the process illustrated in FIG. 1B may be run on all the aggregated data at the end of the day. Therefore, according to an embodiment of the invention, the process in FIG. 1B is run on accumulated data from multiple transactions and therefore processes aggregated transactional data from multiple sources that have been run through the exemplary process as illustrated in FIG. 1A.

At step 110, if a matching pharmacy ID for the prescription transaction exists in the existing event clusters, step 111 follows and no further association passes are performed. For transactions in which a matching pharmacy ID does not exist in the existing event clusters, the further association passes are run in order to match the incoming prescription transactions with event clusters already in the system and the next action is step 111. According to an exemplary embodiment of the invention, step 110 is performed on aggregated transactions that have been provided by multiple sources and collected over the period of a day.

For those transactions that have been matched with a pharmacy ID at 110, sort process 111 sorts the transactions based on a predetermined priority order. Transactions are preferably grouped and ordered by transaction date and time, speeding analysis of transactions to be associated. According to an embodiment, the data can be sorted based on data source, pharmacy, prescription number, prescription fill date, transaction arrival date, and transaction status. For example, data received may be sorted based on the pharmacy ID. In certain embodiments, date and time may be other possible options for the sorting transactions.

At step 118, the reversals within each data source are identified and paired with earlier transactions. If any transactions provided by that data source are found to be reversals of earlier transactions from that data source, they are identified. For example, a transaction reflecting that a patient failed to pick up a prescription (i.e. a reversal) is paired with the transaction in which the physician initially prescribed that medication to the patient, as long as both transactions are provided by a single data source.

At step 119, the system then determines whether the incoming transaction is a claim. A claim is a specific type of data set provided by certain sources that contain comprehensive information regarding a prescription. Based on the data-content and/or identifiers, a claim may be distinguished from a transaction that is purchase data, which is provided by retail stores like pharmacies. Claim information typically includes information including whether the transaction ends in a reversal. If a reversal is indicated by the claim, then any additional required information for that claim can be extracted from its existing event cluster at step 122. A claim indicating that a transaction was a reversal may not include information about a refill number because it was already determined that the claim ended in a reversal. For a complete picture of the prescription event, however, the refill number might be extracted from data provided by a different source but part of the same event cluster at step 122.

If the transaction is not a claim at 119, then the next step is de-duplicating within a data source at 120. Transactions provided by each data source are compared to other transactions from the same data source, and if the transaction is a duplicate of an already-existing transaction, it is marked as such.

At step 121, the transactions within each event cluster are sequenced. Under a preferred embodiment, transactions are chronologically sequenced by time and date to identify the last or final transaction in a cluster. The processes in 131, 136, and 126 are the same as in steps 119, 120, and 121, respectively, except performed on event cluster data that has been run through additional association passes.

The update process in step 123 updates any prescription association tables or databases that are being used to store the information.

If a common pharmacy with the existing transactions cannot be found at 110, then the system uses other keys to determine whether the transaction cluster should be associated with another cluster in the system, starting with the tertiary association pass. The tertiary (112), quaternary (114), and quinary (116) association passes cluster associated transactions based on the pharmacy ID and different combinations of attributes. In other words, the tertiary through quinary association processes look for specific sets of available attributes in the incoming transactions and perform comparisons of selected attributes of the incoming transaction with the attributes in the existing event clusters to determine whether the incoming transaction should be associated with a particular event cluster. If a match is found at any of the association passes (113, 115, 117), the incoming prescription transaction is a match with an event cluster, and the information contained in the transaction is added to that existing event cluster at steps 128, 132, or 135. Also at steps 128, 132, or 135, if a matched transaction is operating with an interim ID, the interim ID is replaced with a permanent ID of the matched event cluster.

According to a preferred embodiment of the present invention, a separate table is used to keep track of associated clusters, and this table is updated with the matched data at steps 128, 132, or 135.

If a match is not found at the last association pass, which according to the current embodiment is the quinary association pass 116, an append process 134 is executed where the unmatched transaction becomes a new cluster and the interim ID becomes a permanent ID. This new cluster is added to the database at step 134.

It is understood by those skilled in the art that, although the example herein utilized a quinary association process, larger or smaller numbers of association processes may be utilized, depending on the needs of the user. For example, if a match is not found within the steps 113, 115, or 117, additional match passes (such as senary, septenary, etc.) are possible before performing an append process.

According to an embodiment of the invention, all the association passes can be run in parallel and the highest priority match can be used.

Table 1 illustrates association keys that are utilized using the association passes shown in FIGS. 1A-1B above. As discussed regarding FIG. 1A, if a pharmacy ID, prescription indicator, and refill code number are available, a first association pass is used to attempt to match that transaction to existing transaction clusters in the system. If a pharmacy ID, product NDC, and prescription fill date are available, a second association pass is used to attempt to match that transaction to an existing event cluster in the system. Under an embodiment of the invention, the values for certain attributes do not have to match exactly but rather within a range. For example, prescription fill date may be matched within a one-day differential.

When a transaction is not matched during the primary or secondary association passes, one or more of the tertiary through senary association passes is carried out, using the keys laid out in Table I, which is provided below:

TABLE I

| | Pri- | Sec- | | | | |
|---|---|---|---|---|---|---|
| | mary | ondary | Tertiary | Quaternary | Quinary | Senary |
| Pharmacy ID | X | X | X | X | X | X |
| Prescription | X | X | | | | |
| Number | | | | | | |
| Product | | X | | | | |
| NDC | | | | | | |
| (10 digit) | | | | | | |
| Prescription | X | X | X | X | X | X |
| Fill Date | | | | | | |
| Drug (Entire | | | X | X | X | X |
| NDC) | | | | | | |
| New or | X | | X | X | | |
| Refill | | | | | | |
| Code | | | | | | |
| Indicator | | | | | | |
| Gender | | | X | | X | |
| Age | | | X | | X | |
| Practitioner | | | | X | | |
| Days Supply | | | | X | | |
| Number of | | | | | | X |
| Refills | | | | | | |
| Authorized | | | | | | |

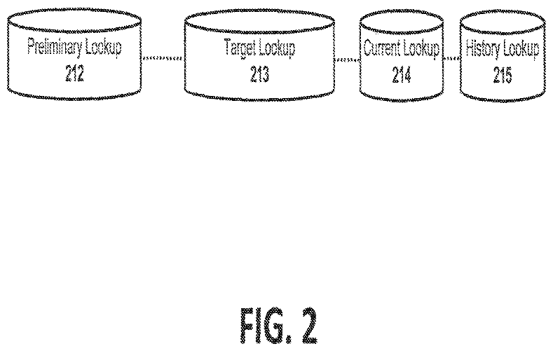

FIG. 2 shows the structures of tables that can be used to store information according to an embodiment of the invention. In this particular embodiment, lookup tables 214 and 215 are used during the disclosed process to store and retrieve attribute data for processing. Specifically, current lookup table 214 is configured to store data for a specific period of time (e.g., 60 days) from a current date relative to a pharmacy. Older lookup data is moved to a history lookup table 215 for long-term storage. It is understood by those skilled in the art that the storage of lookup data can be accomplished using a single storage device, or alternately using multiple storage devices. Lookup table 214 may store transaction attributes related to association passes, and duplicate, partial fill, and reversal processing, as well as prescription anchor data for particular clusters. A storage device is typically a hardware device capable of storing data and comprises two general categories, primary volatile storage devices (e.g., RAM), and a secondary non-volatile storage device (e.g., a hard disk drive or solid state drive).

When utilizing lookup tables for the association passes described above, a preliminary lookup table 212 is loaded with new entries for the current date or batch. The table is then processed to identify transactions where the prescription numbers do not match the numbers of any of the event cluster's anchors. The non-matching transactions are then filtered so that they do not appear with clusters having two unique prescription numbers. The remaining transactions are then grouped according to transaction IDs and dates (preferably oldest date first).

Target lookup table 213 is then loaded to contain the matched transactions, event cluster information and an association pass code. Target lookup table 213 is then filtered so that prescription numbers that do not match anchor numbers and a second prescription number in the cluster are removed. The remaining transactions are then grouped by transaction ID according to date (preferably the oldest date first) and the lowest association pass code. Target lookup table 213 is then populated with the resulting transaction ID, claim ID and association pass code.

Target lookup table 213 is further loaded with new anchor transactions and unmatched transactions (single event clusters) with their transaction IDs as the claim IDs and “0” as the association pass code. Reversal and paid transactions are grouped according to transaction ID and date (preferably the most recent date first). Target lookup table 213 is then loaded with these transactions and their pairs, if available. Duplicate and partial fill transactions, along with their pairs, are also loaded to the target table 213. These transactions are then filtered to remove ones identified as reversal paid pairs.

Of the remaining matches, duplicate transactions that have been matched with pairs from more than two pharmacy prescription dates are identified, and the record status is set for continuously reported/recurring. The remaining matches are then grouped by transaction ID, where partial fill entries are picked over duplicate entries, according to the earliest matching transaction ID pairs. The target table 213 is then loaded with these transactions, their pairs and record status of “duplicate” or “paid.”

Historical association is maintained on a current history table in the ODS for 60 days. As the claim association information ages beyond 60 days from the current process date, the data is moved to a historical history table in the ODS. New data is added to the current history table for a current day's data or batches following the association process where the claim ID and anchor date are equal.

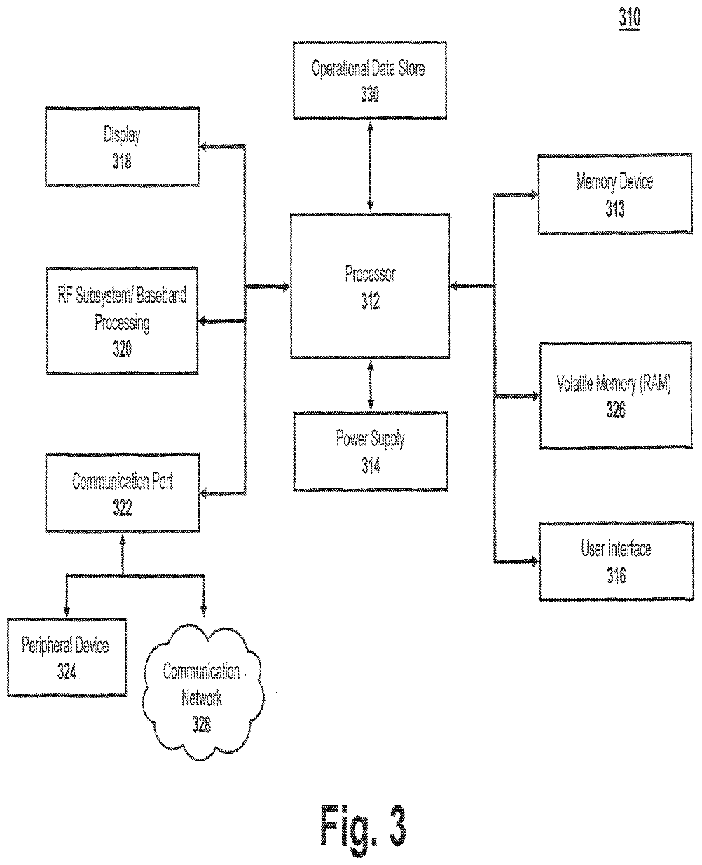

FIG. 3 depicts an exemplary processor-based system 310 that may include one or more memory devices 313 capable of carrying out the present disclosure. The system 310 may be any of a variety of devices such as a computer, pager, cellular phone, personal organizer, control circuit, etc. In a typical processor-based system, one or more processors 312, such as a microprocessor, control the processing of system functions and requests in the system 310. In certain embodiments, various existing processor-based devices may be modified merely by software and/or minor hardware changes to carry out the present disclosure. The system 310 typically includes a power supply 314. For instance, if the system 310 is a portable system, the power supply 314 may advantageously include a fuel cell, permanent batteries, replaceable batteries, and/or rechargeable batteries. The power supply 314 may also include an AC adapter, so the system 310 may be plugged into a wall outlet, for instance. The power supply 314 may also include a DC adapter such that the system 310 may be plugged into a vehicle cigarette lighter, as another example.

Various other devices may be coupled to the processor 312 depending on the functions that the system 310 performs. To illustrate, a user interface 316 may be coupled to the processor 312. The user interface 316 may include buttons, switches, a keyboard, a light pen, a mouse, a digitizer and stylus, and/or a voice recognition system, for instance. A display 318 may also be coupled to the processor 312. The display 318 may include an LCD, an SED display, a CRT display, a DLP display, a plasma display, an OLED display, LEDs, and/or an audio display, for example. Furthermore, an RF sub-system/baseband processor 320 may also be coupled to the processor 312. The RF sub-system/baseband processor 320 may include an antenna that is coupled to an RF receiver and to an RF transmitter (not shown). One or more communication ports 322 may also be coupled to the processor 312. The communication port 322 may be adapted to be coupled, wired or wirelessly, to one or more peripheral devices 324. The one or more peripheral devices 324 may include, for example, a modem, printer, computer, or other auxiliary device. In certain embodiments, the communication port 322 may be enabled for communication, wired or wirelessly, with a communication network 328, such as a local area network, remote area network, intranet, or the Internet, for instance.

The processor 312 may be coupled to an Operational Data Store (“ODS”) 330 capable of providing attributes for use in identification of transactions. The ODS 330 may be a database for integrating data from multiple sources and for standardizing the data to make analysis of the information more efficient. The processor 312 generally controls the system 310 by implementing software programs stored in the memory. The memory is operably coupled to the processor 312 to store and facilitate execution of various programs. For instance, the processor 312 may be coupled to the volatile memory 326 which may include Dynamic Random Access Memory (“DRAM”) and/or Static Random Access Memory (“SRAM”). The volatile memory 326 is typically large so that it can store dynamically loaded applications and data. As described further below, the volatile memory 326 may be configured in accordance with embodiments of the present invention.

The processor 312 may also be coupled to memory device 313. The memory device 313 may include a read-only memory (“ROM”), such as erasable programmable read only memory (“EPROM”), and/or flash memory to be used in conjunction with the volatile memory 326. Similarly, depending on the system configuration, memory device 313 may spread data across one or more servers. The size of the ROM is typically selected to be just large enough to store any necessary operating system, application programs, and fixed data. Additionally, the non-volatile memory may include a high-capacity memory such as a tape or disk drive memory. In an embodiment of the invention, the memory device 313 may include a separate data warehouse used to store data that is generally used on a less frequent basis.

The memory device 313 and volatile memory 326 may store various types of software, such as an operating system or office productivity suite including a word processing application, a spreadsheet application, an email application, and/or a database application. For example, in operation, data may be received from one or more sources and, as received, it may identified as a batch. The batch may then be imaged and stored prior to being processed by, for example, the exemplary process described in FIGS. 1A and 1B, on staging tables. These staging tables may be stored for a predetermined length of time (e.g., ranging from less than a day to indefinitely) in a server. In certain embodiments, after the processing of the data is performed, for example, by the exemplary process described in FIGS. 1A and 1B, the data is stored in a production warehouse and accessed when needed.

Although various embodiments of the present invention have been described with reference to a particular arrangement of parts, features, and the like, these are not intended to exhaust all possible arrangements or features, and indeed many other embodiments, modifications, and variations will be ascertainable to those of skill in the art.

What is claimed is:

1. A processor-implemented method for associating prescription-related data, the method comprising the ordered steps of:
providing at least one processor configured to:
receive incoming batch transaction data from a plurality of sources, said incoming batch transaction data being standardized and stored to an operational data store and comprising a plurality of classifications;
process the classifications in order to obtain attributes for the classifications;
process the attributes to determine matches among the attributes for a respective classification for a predetermined period of time,
wherein the at least one processor matches the attributes via ordered steps of:
a primary association pass, whereby the primary association pass compares attributes reflecting at least one of (1) prescription number, (ii) prescription fill date, or (iii) refill code of the incoming batch transaction data to attributes in existing event clusters; and
a secondary association pass, whereby the secondary association pass compares attributes reflecting at least one of (i) pharmacy ID, (ii) prescription number, (iii) drug identification number, or (iv) prescription fill of the incoming batch transaction data to attributes in said existing event clusters;
wherein, when no match has occurred in either the primary or secondary association pass, the at least one processor matches incoming attribute groups to attributes in existing event clusters via ordered steps of:
a tertiary association pass, whereby the tertiary association pass compares attributes reflecting a tertiary incoming attribute group of the incoming batch transaction data to attributes in said existing event clusters;
a quaternary association pass, whereby the quaternary association pass compares attributes reflecting a quaternary incoming attribute group of the incoming bath transaction data to attributes in said existing event clusters;
a quinary association pass, whereby the quinary association pass compares attributes reflecting a quinary incoming attribute group of the incoming batch transaction data to attributes in said existing event clusters; and
updating existing event clusters to include one more attributes of the incoming batch transaction data when a match with one or more existing event clusters has occurred in the primary, secondary, tertiary, quaternary, or quinary association passes, thereby identifying events associated with the prescription-related data; and
grouping into one or more event clusters the matches for each attribute during the predetermined period of time to identify events associates with the prescription-related data that do not match an existing event cluster,
wherein the tertiary incoming attribute group comprise at least two of the following: prescription fill date, refill code, drug identification number, gender and age;
wherein the quaternary incoming attribute group comprise at least two of the following: prescription fill date, refill code, drug identification number, practitioner and day's supply; and
wherein the quinary incoming attribute group comprise at least two of the following: prescription fill date, refill code and number of refills authorized.

2. The method of claim 1, wherein the events comprise prescribing patterns, payer influences, and patient acceptance of therapy.

3. The method of claim 1, wherein the operational data store is capable of providing attributes for use in identification of transactions.

4. The method of claim 1, wherein the grouping of the matches in the computer system comprises the step of designating one of the plurality of attributes as an anchor to upon which the grouped matches will be based.

5. The method of claim 1, wherein the quinary association attributes comprise pharmacy identification, prescription number, refill code, and a prescription fill date with a specified range.

6. The method of claim 1, wherein the quaternary association attributes comprise pharmacy identification, prescription number, drug identification code, and a prescription fill date within a specified range.

7. The method of claim 1, wherein the quinary association attributes comprise pharmacy identification, prescription fill date, drug identification code, new or refill code, patient gender code, and patient age within a specified range.

8. A computer system for associating prescription-related data, comprising:
a memory;
a communication device operatively coupled to the memory to receive incoming batch transaction data from a plurality of sources, said incoming batch transaction data relating to the prescription-related data, said batch transaction data comprising a plurality of predetermined classifications; and
at least one processor, operatively coupled to the communications device for processing the classifications in order to obtain attributes for the classifications and processing the attributes to determine matches among the attributes for a respective classification for a predetermined period of time;
wherein the at least one processor matches the attributes via ordered steps of:
(a) a primary association pass, whereby the primary association pass compares attributes of (i) prescription number, (ii) prescription fill date, and (iii) refill code of the incoming batch transaction data to attribute in existing event cluster;
(b) a secondary association pass, whereby the secondary association pass compares attributes of (i) parmarch ID, (ii) prescription number, (iii) drug identification number, and (iv) prescription fill of the incoming batch transaction data to attribute in said existing event clusters;
wherein, when no match has occurred in either the primary or secondary association pass, the at least one processor matches incoming attribute groups to attribute in existing event clusters via ordered steps of:
(c) a tertiary association pass, whereby the tertiary association pass compares attributes reflecting a tertiary incoming attribute group of the incoming batch transaction data to attribute in said existing event clusters:
(d) a quaternary association pass, whereby the quaternary association pass compares attributes reflecting a quaternary incoming attribute group of the incoming batch transaction data to attributes in said existing event clusters; and
(e) a quinary association pass, whereby the quinary association pass compares attributes reflecting a quinary incoming attribute group of the incoming batch transaction data to attributes in said existing event clusters;
wherein the at least one processor updates existing clusters to include one more attributes of the incoming batch transaction data when a match with one or more existing event clusters has occurred in the primary, secondary, tertiary, quaternary, or quinary association passes, thereby identifying event associated with the prescription-related data;
wherein the at least one processor groups into one or more event clusters the matches for each attribute during the predetermined period of time to identify event associated with the prescription-related data that do not match an existing event cluster,
wherein the tertiary incoming attribute group comprises at least two of the following: prescription fill date, refill code, drug identification number, gender and age;
wherein the quaternary incoming attribute group comprises at least two of the following: prescription fill date, refill code, drug identification number, practitioner and day's supply; and
wherein the quinary incoming attribute group comprises at least two of the following: prescription fill date, refill code, and number of refills authorized.

9. The computer system of claim 8, wherein tertiary incoming attribute group further comprises pharmacy identification number, data source code, prescription number, and claim status code.

10. The computer system of claim 9, wherein the quaternary incomes attribute group further comprises prescribing patterns, payer influences, and patient acceptance of therapy.

11. The computer system of claim 10, wherein the quinary incoming attribute group pharmacy identification, and prescription number.

12. The computer system of claim 11, wherein the quaternary incoming attribute group further comprises pharmacy identification, and prescription number.

13. The computer system of claim 12, further comprising the step of processing the matches to identify and remove duplicates in the batch transaction data.

14. A computer network for associating prescription-related data, comprising:
at least one memory device;
at least one communication device operatively coupled to the at least one memory device to receive incoming batch transaction data from a plurality of sources, said incoming batch transaction data relating to the prescription-related data, said incoming batch transaction data comprising a plurality of predetermined classifications;
and at least one processor, operatively coupled to the communications device for processing the classifications in order to obtain attributes for the classifications and processing the attributes to determine matches among the attributes for a respective classification for a predetermined period of time;
wherein the at least one processor performs ordered steps of:
(i) processing the attributes by identifying a first matching attributes comprising pharmacy identification, prescription number, refill code, and a prescription fill date within a specified range,
(ii) processing the attributes by identifying a second matching attribute comprising pharmacy identification, prescription number, drug identification code, and a prescription fill date within a specified range,
(iii) processing the attributes by identifying a third matching attribute comprising prescription fill date, refill code, drug identification number, gender and age,
(iv) processing the attributes by identifying a fourth matching attribute comprising fill date, refill code, drug identification number, practitioner and days' supply,
(vii) processing the attributes by identifying a fifth matching attribute comprising prescription fill date, refill code and number of refills authorized,
(viii) updating existing event clusters to include one more attributes of the incoming batch transaction data when a match with one or more existing event clusters has occurred in the first, second, third, fourth or fifth matching attribute processes; and
(ix) grouping into one or more event clusters the matches for each attribute during the predetermined period of time to identify events associates with the prescription-related data that do not match an existing event cluster.
